(19) United States
(12) Patent Application Publication
(10) Pub. No.: US 2009/0156625 A1
(43) Pub. Date: Jun. 18, 2009

(54) Novel Use of a Nicotinic Receptor Agonist 156

(75) Inventors: Sarah Maria Valentine Brockbank, Derbyshire (GB); Maurice Ronald Charles Needham, Leicestershire (GB); Peter Newham, Cheshire (GB); Timothy Martin Piser, Wilmington, DE (US); Jeffrey Scott Smith, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/275,464

(22) Filed: Nov. 21, 2008

Related U.S. Application Data
(60) Provisional application No. 60/989,500, filed on Nov. 21, 2007.
(60) Provisional application No. 61/086,576, filed on Aug. 6, 2008.

(51) Int. Cl. A61K 31/439; A61P 19/02

(52) U.S. Cl. 514/278

(57) ABSTRACT

The invention relates to a novel method of treatment or prophylaxis of osteoarthritis, which comprises administering to a patient in need thereof a therapeutically effective amount of an alpha 7 nicotinic AcetylCholine receptor agonist of Formula (I), or a pharmaceutically acceptable salt thereof:

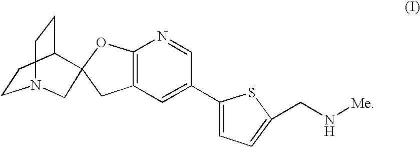

This application claims the benefit under 35 U.S.C. § 119(e) of U.S. Provisional Applications 60/989,500 filed on 21 Nov. 2007 and 61/086,576 filed on 6 Aug. 2008.

### FIELD OF THE INVENTION

This present invention relates to methods for the treatment or prophylaxis of osteoarthritis (OA) using an alpha-7 nicotinic acetylcholine receptor agonist or pharmaceutically acceptable salts thereof.

### BACKGROUND OF THE INVENTION

The loss of articular cartilage is characteristic of the pathology of OA, a debilitating disease of the joints, generally believed to be caused by an imbalance between the synthesis and degradation of articular cartilage matrix. Matrix metalloproteinases (MMPs) have long been considered to play a major regulatory role in maintaining cartilage homeostasis and deregulation of their expression or activity as a result of increased cytokine levels is associated with early degenerative joint disease.

The MMPs play a principle role in the cleavage of matrix macromolecules with the collagenase and stromelysin families of MMPs being of greatest importance to OA as they specifically degrade native collagens and proteoglycans. MMP3 (stromelysin) serves as an activator of latent collagenases and collagenases involved in type II collagen degradation including MMP1, MMP7, MMP8, MMP13 and MMP14 (MT1-MMP). Expression of MMP13 is highly upregulated in disease, in particular late stage disease, and its ability to degrade Type II collagen more effectively than other MMPs suggests a major role for this protease in cartilage degradation (Mitchell et al., 1996; Knauper et al., 1996, Bau et al, 2002).

An additional feature of cartilage degradation concerns the loss of the aggrecans from the extracellular matrix. The reduction of aggrecan content significantly alters the material properties of cartilage that provide much of its load bearing function. Changes in these properties decrease compressive resilience and may contribute to disruption of collagenous organization. Aggrecanase activity results in cleavage of aggrecan at a specific site yielding two neoepitopes, both of which are increased in OA (Lark et al, 1997).

The alpha 7 nicotinic AcetylCholine (α7 nACh) receptor (Gene name: CHRNA7; European Molecular Biology Laboratory (EMBL) Accession Number U40583; Refseq NM_{—}000746) belongs to a family of ligand-gated cation channels which exist as homopentameric and heteropentameric receptors. The α7 nACh receptor itself exists as a homopentameric surface receptor and is expressed by a range of tissues and cell types. The main function of this receptor family is to transmit signals mediated by the neurotransmitter acetylcholine at neuromuscular junctions and in the central and peripheral nervous systems. In addition, α7 nACh receptors are expressed in the hippocampus and play a key role in hippocampal function and memory formation.

Recent evidence has indicated that basic neural pathways also monitor and adjust the inflammatory response. Local inflammation activates an anti-inflammatory response, through stimulation of the vagus nerve by acetylcholine, termed the cholinergic anti-inflammatory pathway. Interestingly, in tissues devoid of innervation there is also evidence of cholinergic mechanisms. Endothelial cells, epithelial cells and lymphocytes express nicotinic acetylcholine receptors and are able to synthesise acetylcholine. Such cells may therefore respond in an autocrine as well as paracrine fashion through acetylcholine receptors (Kawashima et al, 2007; Kurzen et al, 2007; Lips et al, 2007). The inventors have now shown that the α7 nACh receptor is expressed in human articular cartilage. In addition, the inventors have surprisingly found that an α7 nACh receptor agonist is capable of reducing the level of MMP13 expression and aggrecanase activity in cartilage taken from an in vivo mouse model of acute articular inflammation. Given the significance of MMP13 and aggrecanase activity in the OA disease phenotype, the α7 nACh receptor agonist of the present invention is expected to inhibit cartilage degradation and thereby be of use in the treatment or prophylaxis of OA.

Moreover, the inventors have demonstrated that an α7 nACh receptor agonist is capable of reducing the level of MMP13 expression in cartilage taken from an in vivo mouse model of acute articular inflammation at a surprisingly low dose. Since such efficacy has been achieved with very low doses of the α7 nACh receptor agonist, the likelihood of encountering serious side-effects when treating subjects with corresponding doses in a clinical setting is expected to be much reduced.

### BRIEF DESCRIPTION OF THE DRAWINGS

FIG. 1. Human α7nACh receptor Quantative RT-PCR in normal human tissues and human articular cartilage. α7nACh receptor expression was normalised to β-actin RNA.

FIG. 2. Human α7nACh receptor Quantative RT-PCR in normal (post-mortem) and osteoarthritic articular cartilage. Samples include RNA derived from medial and lateral femoral condyles and tibial plateaus, OA1-11 (osteoarthritic cartilage) and PM1-12 (post mortem cartilage). α7nACh receptor expression was normalised to GAPDH RNA.

FIG. 3. Staining with FITC labelled α7nACh receptor antagonist α-bungarotoxin in intact osteoarthritic cartilage. Arrows indicate α7nACh receptor positive chondrocytes (40× magnification).

FIG. 4. The effect of E1 on cartilage extract MMP13 levels in the histamine-induced acute articular inflammation mouse model (1 mg/kg, 0.1 mg/kg b.i.d. for 48 hrs and 1 mg/kg dosed once over the 48 hr study period).

FIG. 5. The effect of E1 on cartilage extract S-GAG levels in the histamine-induced acute articular inflammation mouse model (1 mg/kg, 0.1 mg/kg b.i.d. for 48 hrs and 1 mg/kg dosed once over the 48 hr study period).

FIG. 6. The effect of E1 on cartilage extract MMP13 levels in the histamine-induced acute articular inflammation mouse model dosed at 0.1 mg/kg, 0.001 mg/kg and 0.0001 mg/kg b.i.d. over the 48 hr study period.

FIG. 7. Circulating plasma levels of compound in the mouse following single dose oral administration of E1 at 1 mg/kg and modelled plasma levels for predicted single doses of E1 at 0.1, 0.03, 0.01 and 0.001 mg/kg.

### DETAILED DESCRIPTION OF THE INVENTION

There is therefore provided a method of treatment or prophylaxis of osteoarthritis, which comprises administering to a patient in need thereof a therapeutically effective amount of a compound of Formula (I), namely N-methyl-1-[5-(3′H-spiro[4-azabicyclo[2.2.2]octane-2,2′-furo[2,3-b]pyridin]-5′-yl)-2-thienyl]methanamine, or a pharmaceutically acceptable salt thereof:

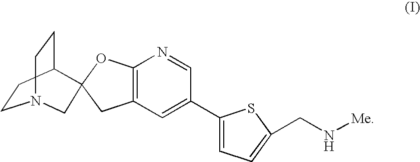

According to another aspect of the invention, there is provided a compound of Formula (I) as defined hereinbefore, or a pharmaceutically-acceptable salt thereof, for use in the treatment or prophylaxis of osteoarthritis.

According to a further aspect of the invention, there is provided the use of a compound of Formula (I) as defined hereinbefore, or a pharmaceutically-acceptable salt thereof, for the preparation of a medicament for the treatment or prophylaxis of osteoarthritis.

According to a further aspect of the invention, there is provided a medicament for the treatment or prophylaxis of osteoarthritis, comprising a compound of Formula (I), or a pharmaceutically acceptable salt thereof, as an active ingredient.

According to a further aspect of the invention, there is provided a method of treatment or prophylaxis of osteoarthritis, which comprises administering to a patient in need thereof a pharmaceutical composition comprising a therapeutically effective amount of a compound of Formula (I) as defined hereinbefore, or a pharmaceutically acceptable salt thereof, and a pharmaceutically acceptable carrier or diluent.

According to a further aspect of the invention, there is provided a pharmaceutical composition comprising a therapeutically effective amount of a compound of Formula (I) as defined hereinbefore and a pharmaceutically acceptable carrier or diluent, for use in the treatment or prophylaxis of osteoarthritis.

In one embodiment of the invention, there is provided a method of treatment or prophylaxis of osteoarthritis which comprises the oral administration to a patient in need thereof of a therapeutically effective amount of a compound of Formula (I) as defined hereinbefore, or a pharmaceutically acceptable salt thereof.

In one embodiment of the invention, there is provided a method of treatment or prophylaxis of osteoarthritis which comprises administration to a patient in need thereof of a therapeutically effective amount of a compound of Formula (I) as defined hereinbefore, or a pharmaceutically acceptable salt thereof, by intra-articular injection.

As used herein, the term “treatment” is intended to have its normal everyday meaning of dealing with a disease in order to entirely or partially relieve one, some or all of its symptoms, or to correct or compensate for the underlying pathology.

As used herein, the term “prophylaxis” is intended to have its normal everyday meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

It is envisaged that for the methods of treatment or prophylaxis of osteoarthritis mentioned herein, which comprise administering a therapeutically effective amount of a compound of Formula (I) or a pharmaceutical composition comprising a therapeutically effective amount of a compound of Formula (I), the compound of Formula (I) or the pharmaceutical composition comprising a therapeutically effective amount of a compound of Formula (I) will be administered to a mammal, more particularly a human being. Similarly, for the uses of a compound of Formula (I) or a pharmaceutical composition comprising a therapeutically effective amount of a compound of Formula (I), for the treatment or prophylaxis of osteoarthritis mentioned herein, it is envisaged that the compound of Formula (I) or the pharmaceutical composition comprising a therapeutically effective amount of a compound of Formula (I), will be administered to a mammal, more particularly a human being.

The compound of Formula (I) defined above may exist in optically active or racemic forms by virtue of the asymmetric carbon atom. Unless stated otherwise, the invention includes in its definition the optically active (R) form of the compound or the racemic form. The synthesis of optically active forms may be carried out by standard techniques of organic chemistry well known in the art, for example by synthesis from optically active starting materials or by resolution of a racemic form. Racemic compounds are drawn herein as flat structures whereas stereospecific compounds and stereospecific intermediates thereof are drawn with the appropriate stereochemistry indicated.

In one embodiment of the invention, the compound of Formula (I) has the (R)-configuration and is thereby a compound of Formula (IA), namely N-methyl-1-{5-[(2R)-3′H-spiro[4-azabicyclo[2.2.2]octane-2,2′-furo[2,3-b]pyridin]-5′-yl]-2-thienyl}methanamine:

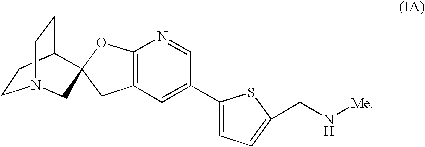

The compound of Formula (I) may also exist in the (S)-configuration and is thereby a compound of Formula (IB), namely N-methyl-1-{5-[(2S)-3′H-spiro[4-azabicyclo[2.2.2]octane-2,2′-furo[2,3-b]pyridin]-5′-yl]-2-thienyl}methanamine:

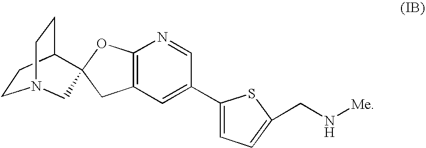

Unless stated otherwise, reference herein to a compound of Formula (I) should be understood to refer equally to a compound of Formula (I) or (IA).

For the uses, methods, medicaments and pharmaceutical compositions mentioned herein, in general, satisfactory results may be obtained when a compound of Formula I is administered orally at a daily dosage of from about 0.00001 mg to about 20 mg/kg of body weight. Such doses may be given in divided doses 1 to 4 times a day or in sustained release form. For man, the total daily dose may be in the range of from 0.0005 mg to 1,400 mg assuming a subject weight of 60-100 kg, more particularly from 0.0005 mg to 1 mg, and unit dosage forms suitable for oral administration may comprise from 0.0001 mg to 1,400 mg of the compound admixed with solid or liquid pharmaceutical carriers, lubricants and diluents.

In one embodiment of the invention, there is provided a method of treatment or prophylaxis of osteoarthritis in a human which comprises the oral administration, to a patient in need thereof, of at least one dose of a therapeutically effective amount of a compound of Formula (IA), or a pharmaceutically-acceptable salt thereof, in the range of 0.00001 to 0.1; 0.00001 to 0.01; 0.00001 to 0.001; 0.0001 to 0.1; 0.0001 to 0.01; or 0.0001 to 0.001 mg/kg of body weight per day.

Alternatively, for the uses, methods, medicaments and pharmaceutical compositions mentioned herein satisfactory results may be obtained when a compound of Formula IA is administered by intra-articular injection.

Thus there is further provided a method of treatment or prophylaxis of osteoarthritis in a human which comprises the administration by intra-articular injection, to a patient in need thereof, of at least one dose of a therapeutically effective amount of a compound of Formula (IA), or a pharmaceutically acceptable salt thereof, in the range of 0.00001 to 0.1; 0.00001 to 0.01; 0.00001 to 0.001; 0.0001 to 0.1; 0.0001 to 0.01; or 0.0001 to 0.001 mg/kg of body weight per day.

In another embodiment of the invention, there is provided a compound of Formula (IA), or a pharmaceutically-acceptable salt thereof, for use in the treatment or prophylaxis of osteoarthritis wherein the treatment comprises the administration of at least one dose of a compound of Formula (IA), or a pharmaceutically-acceptable salt thereof, in the range of 0.00001 to 0.1; 0.00001 to 0.01; 0.00001 to 0.001; 0.0001 to 0.1; 0.0001 to 0.01; or 0.0001 to 0.001 mg/kg of body weight per day.

In a further embodiment of the invention, there is provided the use of a compound of Formula (IA), or a pharmaceutically-acceptable salt thereof, for the preparation of a medicament for the treatment or prophylaxis of osteoarthritis wherein the treatment or prophylaxis comprises the administration of at least one dose of a compound of Formula (IA), or a pharmaceutically-acceptable salt thereof, in the range of 0.00001 to 0.1; 0.00001 to 0.01; 0.00001 to 0.001; 0.0001 to 0.1; 0.0001 to 0.01; or 0.0001 to 0.001 mg/kg of body weight per day.

A compound of Formula I, or a pharmaceutically acceptable salt thereof, may be used on its own or in the form of appropriate medicinal preparations for enteral or parenteral administration. There is therefore provided a pharmaceutical composition including preferably less than 80% and more preferably less than 50% by weight of a compound of Formula I in admixture with an inert pharmaceutically-acceptable diluent, lubricant or carrier.

Examples of diluents, lubricants and carriers are
for tablets and dragees: lactose, starch, talc, stearic acid;
for capsules: tartaric acid or lactose;
for injectable solutions: water, alcohols, glycerin, vegetable oils;

for suppositories: natural or hardened oils or waxes.

According to a further aspect of the invention, there is therefore provided a pharmaceutical composition comprising 0.0001 to 10; 0.0001 to 1.9; 0.0001 to 1; 0.0001 to 0.1; 0.0001 to 0.01; 0.0001 to 0.001; 0.001 to 10; 0.001 to 1.9; 0.001 to 1; 0.001 to 0.1; 0.001 to 0.01; 0.01 to 10; 0.01 to 1.9; 0.01 to 1; or 0.01 to 0.1 mg of a compound of Formula (IA), or a pharmaceutically-acceptable salt thereof, as defined hereinbefore in combination with a pharmaceutically acceptable carrier or diluent.

In one embodiment of the invention, there is provided a pharmaceutical composition comprising 0.001 to 0.1 mg of a compound of Formula (IA), or a pharmaceutically-acceptable salt thereof, as defined hereinbefore in combination with a pharmaceutically acceptable carrier or diluent.

In a further embodiment of the invention, there is provided a medicament for the treatment or prophylaxis of osteoarthritis comprising 0.0001 to 10; 0.0001 to 1; 0.0001 to 0.1; 0.0001 to 0.01; 0.0001 to 0.001; 0.001 to 10; 0.001 to 1; 0.001 to 0.1; 0.001 to 0.01; 0.01 to 10; 0.01 to 1; or 0.01 to 0.1 mg of a compound of Formula (IA), or a pharmaceutically-acceptable salt thereof, as an active ingredient.

A compound of Formula I, a pharmaceutically acceptable salt thereof, or a pharmaceutical composition or formulation comprising a compound of Formula I, may be administered concurrently, simultaneously, sequentially or separately with another pharmaceutically active compound or compounds listed below.

A non-steroidal anti-inflammatory agent (hereinafter NSAID) including a non-selective cyclo-oxygenase COX-1/COX-2 inhibitor whether applied topically or systemically (such as piroxicam, diclofenac, propionic acid such as naproxen, flurbiprofen, fenoprofen, ketoprofen or ibuprofen, fenamate such as mefenamic acid, indomethacin, sulindac or azapropazone, pyrazolone such as phenylbutazone, a salicylate such as aspirin); a selective COX-2 inhibitor (such as meloxicam, celecoxib, rofecoxib, valdecoxib, lumarocoxib, parecoxib or etoricoxib); a cyclo-oxygenase inhibiting nitric oxide donor (CINOD); a glucocorticosteroid; methotrexate; leflunomide; hydroxychloroquine; d-penicillamine; auranofin or other parenteral or oral gold preparation; an analgesic; diacerein; an intra-articular therapy such as a hyaluronic acid derivative; or a nutritional supplement such as glucosamine.

A cytokine, or agonist or antagonist of cytokine function, (including an agent which acts on a cytokine signalling pathway such as a modulator of the SOCS system) including alpha-, beta-, or gamma-interferons; insulin-like growth factor type I (IGF-1); interleukin (IL) including IL1 to 17, and interleukin antagonist or inhibitor such as anakinra; a tumour necrosis factor alpha (TNF-α) inhibitor such as anti-TNF monoclonal antibody (for example infliximab; adalimumab, and CDP-870) and TNF receptor antagonist including immunoglobulin molecule (such as etanercept) and a low-molecular-weight agent such as pentoxyfylline.

A monoclonal antibody targeting B-Lymphocytes (such as CD20 (rituximab), MRA-aIL16R and T-Lymphocytes, CTLA4-Ig, HuMax I1-15).

A modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

An inhibitor of matrix metalloprotease (MMP), such as a stromelysin, a collagenase, or a gelatinase, as well as aggrecanase; for example collagenase-1 (MMP-1), collagenase-2 (MMP-8), collagenase-3 (MMP-13), stromelysin-1 (MMP-3), stromelysin-2 (MMP-10), stromelysin-3 (MMP-11), MMP-9 or MMP-12, including an agent such as doxycycline. A leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; a 2,6-di-tert-butylphenolhydrazone; a methoxytetrahydropyran such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, or BAY x 1005.

A receptor antagonist for leukotrienes (LT) B4, LTC4, LTD4, and LTE4. selected from the group consisting of a phenothiazin-3-1 such as L-651,392; an amidino compound such as CGS-25019c; a benzoxalamine such as ontazolast; a benzenecarboximidamide such as BIIL 284/260; or a compound such as zafirlukast, ablukast, montelukast, pranlukast, verlukast (MK-679), RG-12525, Ro-245913, iralukast (CGP 45715A), or BAY x 7195. A phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

A histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

A proton pump inhibitor (such as omeprazole) or a gastroprotective histamine type 2 receptor antagonist.

An antagonist of the histamine type 4 receptor.

An alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

An anticholinergic agent including muscarinic receptor (M1, M2, and M3) antagonist such as atropine, hyoscine, glycopyrrrolate, ipratropium bromide, tiotropium bromide, oxitropium bromide, pirenzepine or telenzepine.

A beta-adrenoceptor agonist (including beta receptor subtypes 1-4) such as isoprenaline, is salbutamol, formoterol, salmeterol, terbutaline, orciprenaline, bitolterol mesylate, pirbuterol or indacaterol, or a chiral enantiomer thereof.

A chromone, such as sodium cromoglycate or nedocromil sodium.

A glucocorticoid, such as flunisolide, triamcinolone acetonide, beclomethasone dipropionate, budesonide, fluticasone propionate, ciclesonide or mometasone furoate.

An agent that modulates a nuclear hormone receptor such as a PPAR.

An immunoglobulin (Ig) or Ig preparation or an antagonist or antibody modulating Ig function such as anti-IgE (for example omalizumab).

Another systemic or topically-applied anti-inflammatory agent, such as thalidomide or a derivative thereof, a retinoid, dithranol or calcipotriol.

An aminosalicylate or a sulfapyridine such as sulfasalazine, mesalazine, balsalazide or olsalazine; an immunomodulatory agent such as a thiopurine, or a corticosteroid such as budesonide.

An antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

A cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

A CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

An agent for the treatment of acute or chronic pain, such as a centrally or peripherally-acting analgesic (for example an opioid or derivative thereof), carbamazepine, phenyloin, sodium valproate, amitryptiline or other anti-depressant agent, paracetamol, or a non-steroidal anti-inflammatory agent.

A parenterally or topically-applied (including inhaled) local anaesthetic agent such as lignocaine or a derivative thereof.

An anti-osteoporosis agent including a hormonal agent such as raloxifene, or a biphosphonate such as alendronate.

An agent which is a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2×7; (xxvii) inhibitor of transcription factor activation such as NFkB, API, or STATS; or (xxviii) a glucocorticoid receptor modulator (such as an agonist).

A therapeutic agent for the treatment of cancer, for example:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);

(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;

(iii) an agent which inhibits cancer cell invasion (for example a metalloproteinase inhibitor like marimastat or an inhibitor of urokinase plasminogen activator receptor function);

(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as \ddot{N}-(3-chloro-4-fluorophenyl)-7-methoxy-6-(3-morpholinopropoxy)quinazolin-4-amine (gefitinib, AZD1839), \ddot{N}-(3-ethynylphenyl)-6,7-bis(2-methoxyethoxy)quinazolin-4-amine (erlotinib, OSI-774) or 6-acrylamido-\ddot{N}-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;

(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);

(vi) a vascular damaging agent such as combretastatin A4, or a compound disclosed in WO 99/02166, WO 00/40529, WO 00/41669, WO 01/92224, WO 02/04434 or WO 02/08213;

(vii) an agent used in antisense therapy, for example one directed to one of the targets listed above, such as ISIS 2503, an anti-ras antisense;

(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or

(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies.

Such combination products employ the compound of Formula (I) within the dosage range described herein and the other pharmaceutically active compound or compounds within approved dosage ranges and/or the dosage described in the publication reference.

Under certain conditions, a compound of Formula (I) may form an acid addition salt. Acid addition salts of the compound of Formula I include salts of mineral acids, for example the hydrochloride and hydrobromide salts; and salts formed with organic acids such as formate, acetate, maleate, benzoate, tartrate, and fumarate salts. Acid addition salts of a compound of Formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

It will be understood that a compound of the present invention may exist in solvated, for example hydrated, as well as unsolvated forms. It is to be understood that the present invention encompasses all such solvated forms of the compound of Formula (I) which are agonists at the α7 nAChR.

A compound of Formula (I) may also be administered in the form of a prodrug which is broken down in the human or animal body to give a compound of Formula (I). Examples of prodrugs include in vivo hydrolysable esters of a compound of Formula (I). Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);

b) A Textbook of Drug Design and Development, edited by Krogsgaard-Larsen and H. Bundgaard, Chapter 5 “Design and Application of Prodrugs”, by H. Bundgaard p. 113-191 (1991);

c) H. Bundgaard, Advanced Drug Delivery Reviews, 8, 1-38 (1992);

d) H. Bundgaard, et al., Journal of Pharmaceutical Sciences, 77, 285 (1988); and N. Kakeya, et al., Chem Pharm Bull, 32, 692 (1984).

### Example 1

### N-methyl-1-{5-[(2R)-3′H-spiro[4-azabicyclo[2.2.2]octane-2,2′-furo[2,3-b]pyridin]-5′-yl]-2-thienyl}methanamine (E1)

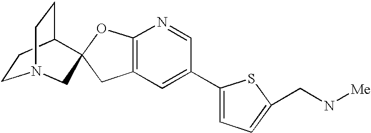

The compound of Example 1 may be prepared according to Scheme 1 as detailed below.

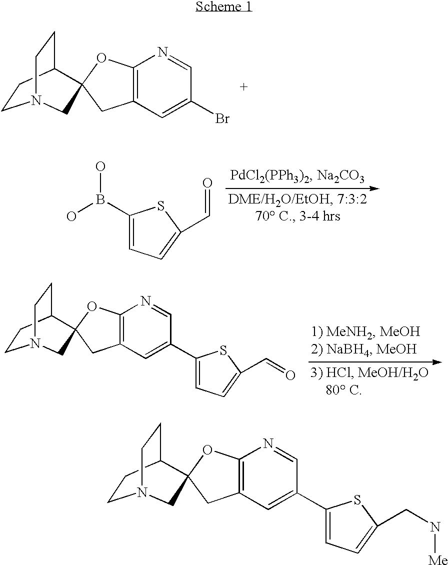

### a) 5-[(2R)-3′H-spiro[4-azabicyclo[2.2.2]octane-2,2′-furo[2,3-b]pyridin]-5′-yl]thiophene-2-carbaldehyde

To a stirred solution of (2R)-5′-bromo-3′H-spiro[4-azabicyclo[2.2.2]octane-2,2′-furo[2,3-b]pyridine] (3.2 g, 10.8 mmol) and (5-formyl-2-thienyl)boronic acid (3.37 g, 21.6 mmol) in 150 mL DME (dimethoxyethane)/H₂O/EtOH (7:3:2), powdered Na₂CO₃ (4.58 g, 43.2 mmol) was added. (The synthesis of (2R)-5′-bromo-3′H-spiro[4-azabicyclo[2.2.2]octane-2,2′-furo[2,3-b]pyridine] is described in WO 99/03859 A1). The resulting mixture was purged with N₂ at room temperature for 15 min, before the addition of dichloro[bis(triphenylphosphoranyl)]palladium (380 mg, 0.54 mmol). The reaction mixture was purged with N₂ for another 15 min, before being heated in a 70° C. oil bath under N₂ for 3-4 h. The reaction mixture was cooled and then concentrated under reduced pressure. The solid residue was treated with 150 mL CHCl₃, and the resulting suspension was filtered through a short pad of diatomaceous earth (the filter cake was washed with ˜100 mL CHCl₃). The combined filtrate was concentrated under reduced pressure. The blackish solid was taken into 0.5 M HCl (100-150 mL) and EtOAc (˜100 mL). The acidic aqueous layer was separated and the blackish organic layer was washed further with 0.5 M HCl (2×50 mL). The combined aqueous layers were then basified with 1 M NaOH to pH ˜12, and extracted with CHCl₃ (100 mL, then 2×50 mL). The CHCl₃ extracts were dried over MgSO₄, filtered, and concentrated to give 3.5 g (99%) of 5-[(2R)-3H-spiro[4-azabicyclo[2.2.2]octane-2,2′-furo[2,3-b]pyridin]-5′-yl] thiophene-2-carbaldehyde. ¹H NMR (500 MHz, CDCl₃) δ 1.50 (dddd, J=12.8, 10.7, 6.7, 2.3 Hz, 1H), 1.68-1.72 (m, 2H), 2.03 (t, J=2.9 Hz, 1H), 2.22-2.26 (m, 1H), 2.78-2.95 (m, 3H), 2.97 (dd, J=14.7, 2.1 Hz, 1H), 3.01-3.05 (m, 1H), 3.08 (d, J=16.5 Hz, 1H), 3.40 (dd, J=14.7, 1.6 Hz, 1H), 3.48 (d, J=16.5 Hz, 1H), 7.29 (d, J=4.0 Hz, 1H), 7.68 (d, J=2.2 Hz, 1H), 7.72 (d, J=4.0 Hz, 1H), 8.36 (d, J=2.2 Hz, 1H), 9.88 (s, 1H); MS ES⁺ m/z 327 (M+H⁺).

b) To a stirred suspension of 5-[(2R)-3H-spiro[4-azabicyclo[2.2.2]octane-2,2′-furo[2,3-b]pyridin]-5′-yl]thiophene-2-carbaldehyde (1.5 g, 4.6 mmol) in 40 mL MeOH, 9.2 mL methylamine methanolic solution (18.4 mmol) was added. The clear solution was stirred at room temperature for 0.5 h before the addition of NaBH₄ (524 mg, 3.0 equiv) as one portion. The reaction mixture was stirred at room temperature for 1 h. The solution was concentrated under reduced pressure to give a black residue, which was dissolved in 60 mL MeOH and treated with 30 mL 4.0 M HCl. The resulting solution was heated at 80° C. for 1-2 h. MeOH was removed under reduced pressure. The resulting acidic aqueous solution was basified by slow addition of conc. aqueous NaOH until pH>12 and extracted with CHCl₃ (3×50 mL). The blackish organic layers were dried over MgSO₄, filtered and concentrated to give a black residue, which was then purified by short-packed silica gel column (5% 7 M NH₃/MeOH in CHCl₃) to give a yellowish solid, which was washed with minimum amount of Et₂O (˜20 mL) to remove minor impurities. Yield: 1.2 g (76%); ¹H NMR (500 MHz, CDCl₃) δ 1.48 (ddd, J=12.8, 10.7, 6.6, 2.3 Hz, 1H), 1.67-1.70 (m, 2H), 2.02 (t, J=2.9 Hz, 1H), 2.22-2.27 (m, 1H), 2.50 (s, 3H), 2.77-2.93 (m, 3H), 2.96 (dd, J=14.7, 2.1 Hz, 1H), 3.01-3.07 (m, 1H), 3.04 (d, J=16.2 Hz, 1H), 3.39 (dd, J=14.7, 1.8 Hz, 1H), 3.43 (d, J=16.2 Hz, 1H), 3.93 (s, 1H), 6.87 (d, J=3.6 Hz, 1H), 7.01 (d, J=3.6 Hz, 1H), 7.59 (d, J=2.2 Hz, 1H), 8.23 (d, J=2.2 Hz, 1H); MS ES⁺ m/z=342 (M+H⁺).

### Example 2

### Alpha 7 Nicotinic Acetyl Choline Receptor (CHRNA7) Expression in Chondrocytes and Cartilage

### Cartilage RNA Extraction

Human articular cartilage samples from osteoarthritic (OA) and post-mortem (PM) donors (normal, no history of OA) were obtained after prior ethical approval. Cartilage was carved from all parts of the knee and pooled. The cartilage was snap frozen and ground under liquid nitrogen using a Glen Creston Spex mill. RNA was extracted from the ground cartilage using a standard TRIzol extraction method (Invitrogen) following manufacturer's protocols. The RNA was purified using a Qiagen RNeasy minicolumn (Qiagen) and treated with DNase. RNA was quantified using an Agilent Bioanalyser 2100 with the RNA Nano6000 chip.

### Taqman™ Methodology

TaqMan real-time quantitative polymerase chain reaction (PCR) assay was performed on an ABI Prism 7700 Sequence Detection System, according to the manufacturer's protocol (Applied Biosystems). TaqMan RT-PCR assay primers and probes were obtained from Assays on Demand (Applied Biosystems).

25 ng of RNA was mixed with a 20× Assay on Demand and Taqman Quantitect Probe Master-Mix and RT enzyme mix (Qiagen). Samples were incubated for an initial reverse transcription reaction at 50° C. for 30 minutes and then at 95° C. for 15 minutes, followed by 40 cycles at 95° C. for 15 seconds and 60° C. for 1 minute. Relative quantitation of target RNA was carried out using SDS v1.9 software and relative expression levels calculated using a standard curve method and normalised to a housekeeping gene (GAPDH or 18s).

### Results

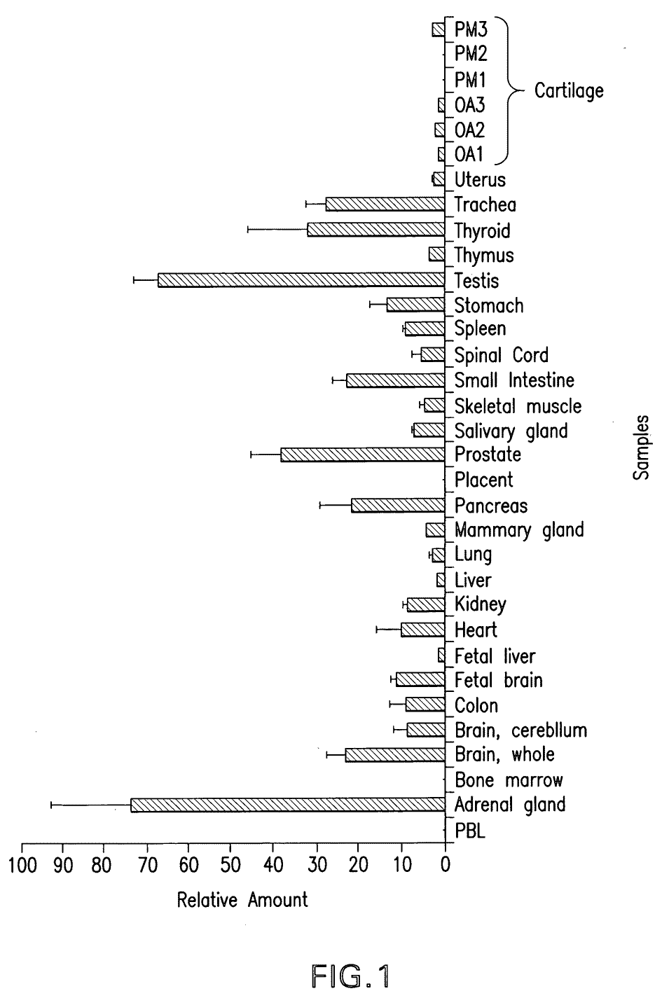

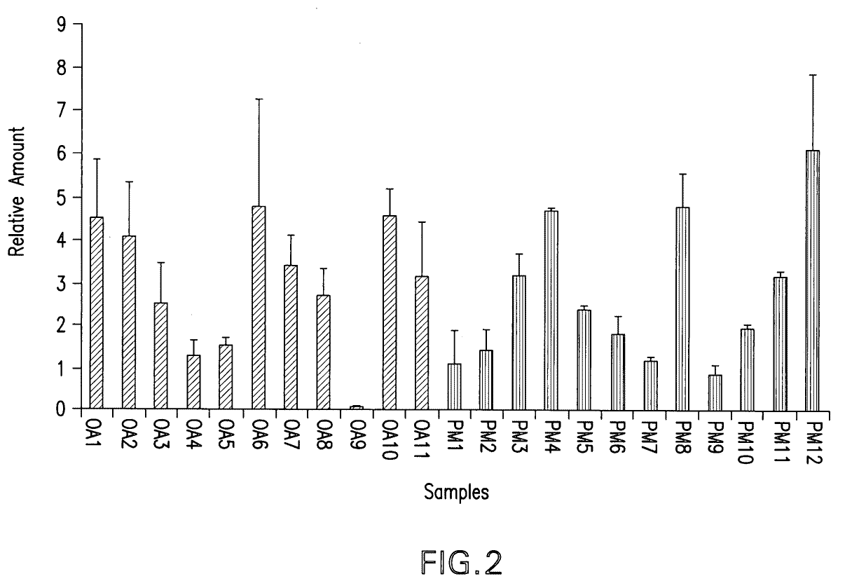

Quantitative RT-PCR (Taqman) demonstrated that the α7 nACh receptor was widely expressed in a variety of human tissues with low levels of expression of α7 nACh receptor mRNA in OA and PM cartilage (FIG. 1). Analysis of expression levels in a panel of osteoarthritic cartilage samples (taken from patients undergoing total knee replacement) and normal cartilage taken post mortem revealed that there was no differential expression of α7 nACh receptor in OA cartilage versus normal (PM) cartilage (FIG. 2).

### Example 3

### Immunohistochemistry

The degree of inhibition of collagen breakdown in human cartilage in response to treatment with an agonist of the α7 nACh receptor, for example N-methyl-1-{5-[(2R)-3′H-spiro[4-azabicyclo[2.2.2]octane-2,2′-furo[2,3-b]pyridin]-5′-yl]-2-thienyl}methanamine (E1), could be assessed using the following method.

5 mm cartilage biopsies are obtained from human OA or PM donors and cultured ex vivo in explant culture media (phenol red-free DMEM (Dulbecco's Modified Eagle Medium; Gibco) with 1% non-essential amino acids (Gibco), 1% PSG (Penicillin/Streptomycin/Glutamine; Sigma), 0.1% Gentamycin-sulphate solution (Sigma) and 1% Amphotecerin-B (Sigma)) in 48-well tissue culture plates at 37° C., 5% CO₂, 21% O₂). Biopsy samples are then treated with an agonist of the α7 nACh receptor, optionally in the presence of Interleukin 1β and Oncostatin M in order to increase endogenous levels of collagen breakdown. Following treatment the biopsies are snap frozen in OCT Embedding Medium (Raymond Lamb) and stored at −80° C. Frozen 7 μm sections are cut and mounted onto Superfrost+slides (VWR). Sections are then fixed in freshly prepared 4% paraformaldehyde for 5 minutes. Endogenous preoxidase is quenched using 3% H₂O₂ (Aldrich) in methanol (30 min). Antigen retrieval is carried out with chondroitinase ABC (Sigma: 0.0125 units per 50 μl chondroitinase buffer as per manufacturers data sheet) for 90 minutes at 37° C. Sections are washed and protein blocked in 20% sheep serum (Dako in TBS/0.05% Tween 20/1% BSA, 60 min, room temp.). Rabbit anti-Hst1 antibody (1.1 ng/μg, AstraZeneca) is incubated overnight at 4° C. and human adsorbed rabbit IgG (1.1 μg/ml; Dako) used as a control. Detection is carried out using 1:400 biotinylated sheep anti-rabbit secondary antibody (Serotec) for 30 minutes at room temperature, amplification using Vectastain ABC kit (Vector Labs; according to manufacturers instructions) and visualisation using 3,3-diaminobenzidine (DAB; Dako) with a haematoxylin counterstain. Sections are dehydrated to xylene and mounted and the levels of collagen breakdown between samples compared.

### Bungarotoxin Methodology

Localisation of the α7 nACh receptor in OA articular cartilage was assessed according to the following method.

Frozen 7 μm sections of OA articular cartilage were cut and mounted on Superfrost+slides (VWR). Sections were allowed to air dry at room temperature for 30 minutes prior to staining. Sections were unfixed for the staining process. Slides were washed for 10 minutes in PBS and then were permeabilized using 0.5 mg/ml bovine testicular hyaluronidase in PBS (Sigma) for 20 minutes at room temperature. Non-specific autofluorescence of the cartilage was reduced by a 10-minute stain at room temperature with 0.1% Toluidine Blue. Sections were washed in water until excess toluidine blue stain was removed. Sections were blocked for 1-hour with PBS+0.5% Triton X-100+0.5% normal goat serum. 100 nM α-bungarotoxin (FITC-labelled, Invitrogen) was then applied to the sections diluted in PBS+0.5% Triton X-100+0.5% normal goat serum. Staining was overnight at 4° C. After washing and blotting dry, sections were mounted using Invitrogen ProlongGold+DAPI (4′,6-diamidino-2-phenylindole; a fluorescent stain that binds strongly to DNA).

### Results

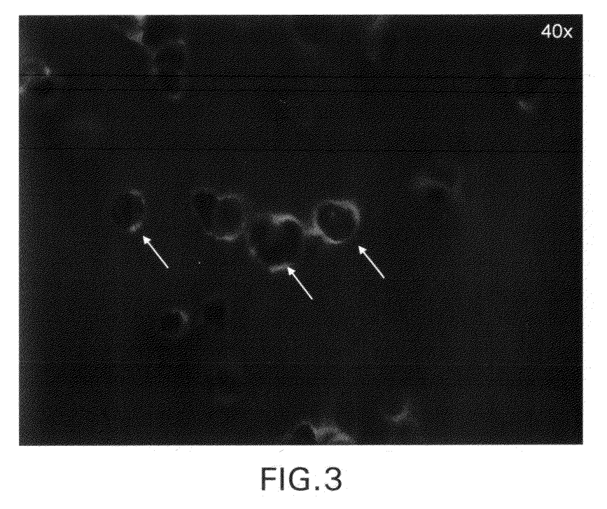

Localisation of a 7 nACh receptor expression in osteoarthritic and normal cartilage was established using specific staining with FITC labelled α-bungarotoxin. α-Bungarotoxin is a selective antagonist of α7nACh receptor that binds to the acetylcholine receptor. α7 nACh receptor was found to be expressed by chondrocytes throughout the full thickness of the cartilage. FIG. 3 shows positive α-bungarotoxin staining indicative of α7nACh receptor chondrocyte expression in intact OA full thickness cartilage.

### Example 4

### In Vivo Pharmacodynamic Model

### Histamine Induced Acute Articular Inflammation in Mouse

At 1-hour post oral treatment with either vehicle hydroxypropyl methylcellulose (HPMC) or E1, C57B16 mice were anaesthetized under isofluorane by inhalation. An H1 specific receptor antagonist desloratadine (5 mg/kg) was used as a positive control. The left hind knee was shaved, dampened with 70% ethanol and injected intra articularly into the knee joint (6 μl volume) with either saline or histamine (5 μM) using a glass Hamilton syringe and 30-gauge needle. Mice were allowed to recover in their home cage and kept on study for 48 hours post injection and dosed with compound.

### Mouse Cartilage Extraction

After synovial lavage, the joint was disarticulated and cartilage was removed with a scalpel from the tibial plateau & femoral condyle, taking care to minimise the amounts of bone. Cartilage samples were weighed and snap frozen. The cartilage from individual animals was extracted in 200 μl high salt buffer (HSB) overnight at 4° C. with gentle agitation.

Samples were centrifuged at 10000 rpm for 10 min and the HSB cartilage extract decanted and stored at −80° C. prior to analysis.

MMP13 fluorokine ELISA (96-Well Plate Format)

HSB cartilage extracts were tested in duplicate at a 1:5 dilution. There were no deviations from the kit instructions (F13M00, R&D Systems) and the plate was read using an excitation wavelength of 320 nm and an emission wavelength of 405 nm.

### Sulphated Glycosaminoglycan (S-GAG) Analysis (DMMB Assay)

HSB cartilage extracts were digested with hyaluronidase (110 units/ml) 37° C. for 3 hrs followed by papain digestion at 60° C. overnight. Digested samples were then centrifuged at 12000 g for 15 minutes. A 5 μl sample in duplicate was transferred to a 384 well plate for analysis. 40 μl DMMB (dimethyl methylene blue, NBS biologicals) was added and gently mixed. The plate was read immediately at 532 nm and 620 nm wavelengths and data calculated as O.D 532 nm/O.D 620 nm minus background. Levels of S-GAG were normalised for wet weight of cartilage.

### Results

Agonism at the α7 nACh Receptor Mediates Effects on Induction of MMP Release and Aggrecanase Activity In Vivo

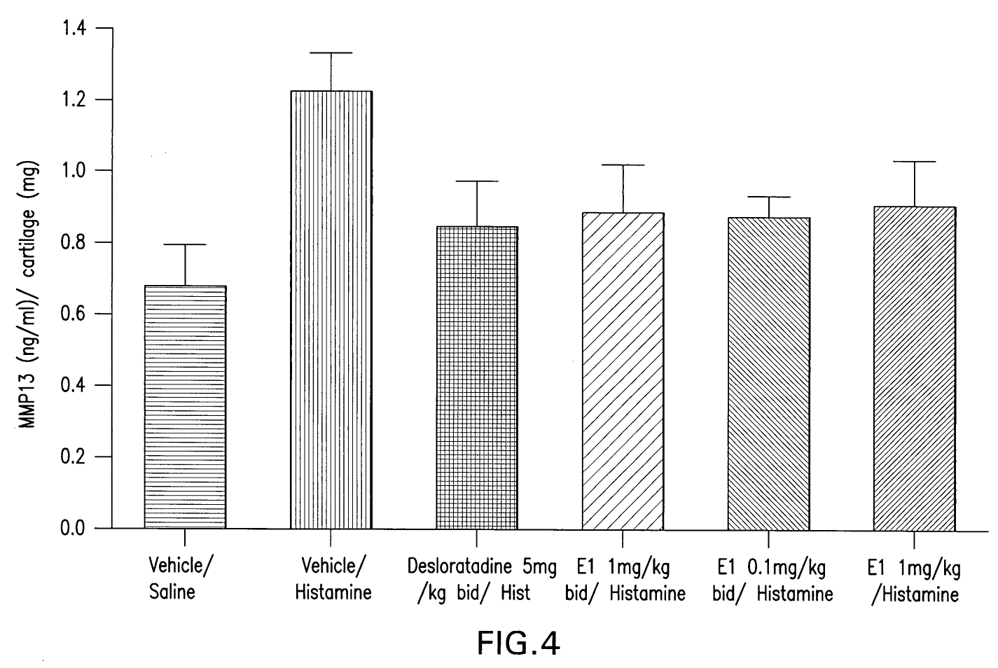

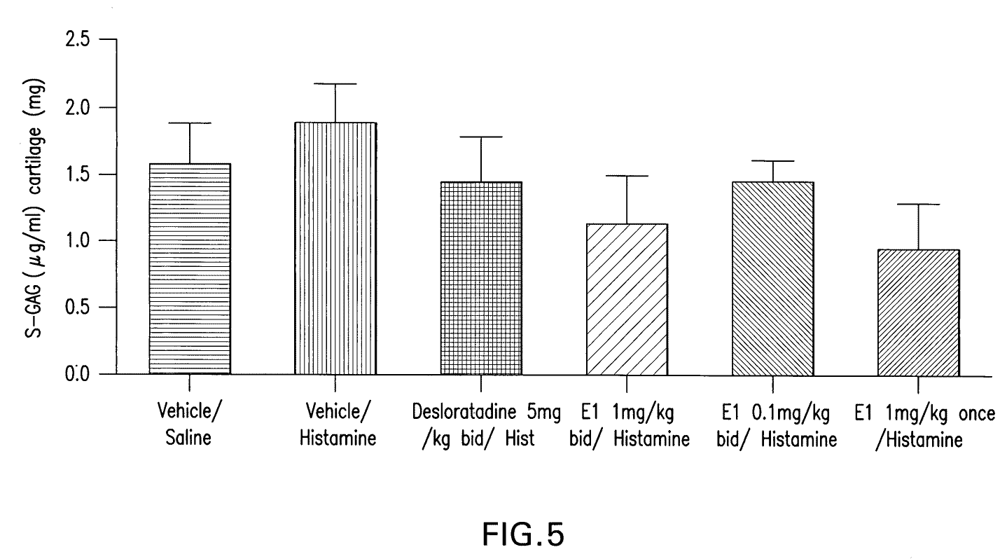

To demonstrate α7nACh receptor functional validation in vivo, C57B16 mice were challenged intra-articularly and treated with E1 at 1 mg/kg, 0.1 mg/kg “bis in die” (b.i.d.) (twice a day) for 48 hrs or 1 mg/kg once over the 48 hr study period. Analysis of the extracted tibial cartilage by ELI SA showed decreased levels of MMP13 with all three agonist treatment groups indicative of reduced collagenase activity in response to E1 (FIG. 4). S-GAG cartilage levels were also significantly reduced with 1 mg/kg α7 nACh receptor (b.i.d. and single dose), and to a lesser extent with 0.1 mg/kg group, indicative of reduced aggrecanase activity in response to E1 (FIG. 5).

Low Doses of α7 nACh Receptor Agonist Mediate Effects on Induction of MMP Release In Vivo

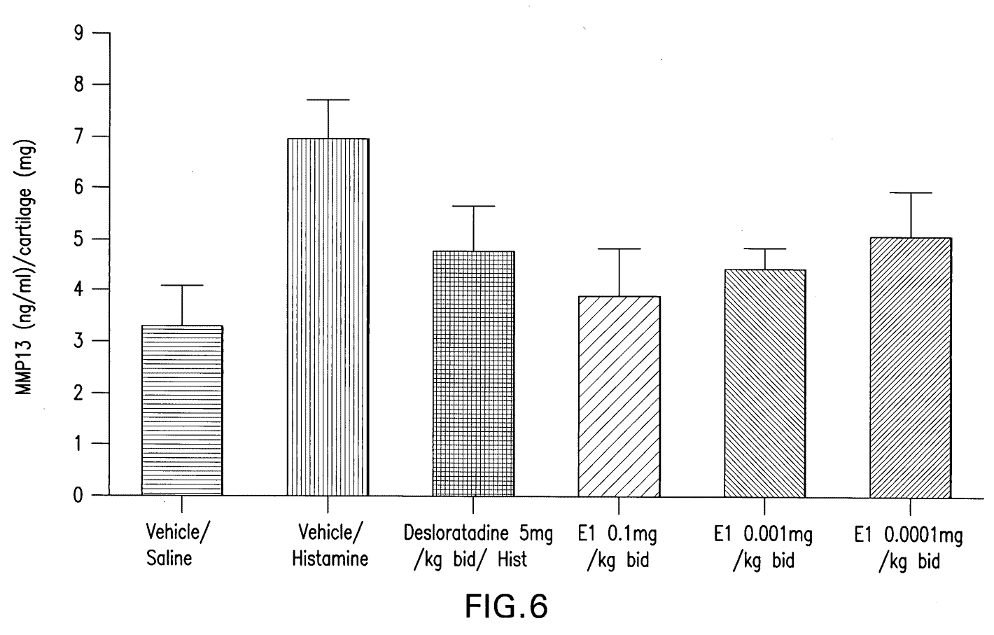

In a further study, C57B16 mice were challenged intra-articularly and treated with E1 at 0.1 mg/kg, 0.001 mg/kg and 0.0001 mg/kg b.i.d. for 48 hrs. Analysis of the extracted tibial cartilage by ELISA again showed decreased levels of MMP13 at all agonist concentrations (FIG. 6).

### Example 5

### Pharmacokinetic (PK) Studies

### Dosing Regime

Mice were weighed and orally dosed according to weight at a dose volume of 10 ml/kg (dosed at 0.1 mg/kg, 0.001 mg/kg and 0.0001 mg/kg b.i.d. for 48 hrs). The PK and low dose pharmacodynamic studies were performed in the same group of animals.

### PK Micro-Sampling

Individual mice were repeatedly sampled at various timepoints over the 48 hr duration of the study by taking small volumes of blood by micro-sampling technique. After heating and mild restrain behind a Perspex stand, the tip of the tail was pricked with a 24 gauge needle and whole blood collected into an EDTA 20 μl glass micro sampling tubes. The 20 μl blood sample was then mixed with 80 μls of distilled water and frozen until analysis.

### Terminal PK Sampling

At study termination the mice were culled using CO₂ gas. A blood volume of about 0.4 mL was obtained by cardiac puncture. A volume of whole blood was then mixed with distilled water to lyse the cells (ratio 1:1) and stored for analysis.

### PK Analysis

E1 was analysed by liquid chromatography tandem mass spectrometry (LC-MS/MS) with limits of quantification at 1-5 ng/ml (3-115 nM).

### PK Extrapolation

As doses of E1 were very low, circulating levels could not be detected in many animals, although some levels could be detected following twice daily dosing at 48 hours indicating some accumulation consistent with a long T_{1/2}. In order to gain confidence in predicting circulating levels, mouse PK at 1 mg/kg oral dosing was subsequently carried out. Circulating levels for lower doses were extrapolated from these data.

### Results

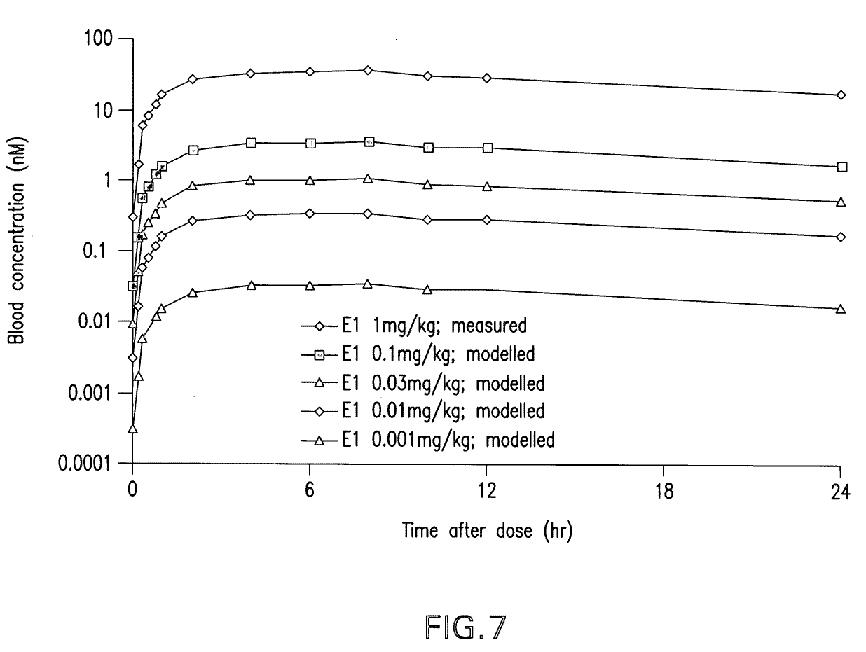

Circulating plasma levels of compound found at various time points following single dose oral administration of E1 to the mouse at 1 mg/kg, along with modelled plasma levels for predicted doses at 0.1, 0.03, 0.01 and 0.001 mg/kg are shown in Table1 and FIG. 7. Blood concentrations are given in nM. AUC and Cmax PK parameters obtained from the actual and modelled circulating plasma levels are shown in the final two rows of Table 1.

TABLE 1

| | | Modelled data (FREE nM) | | | |
|---|---|---|---|---|---|
| Time after | Average | | | | 0.001 |
| dose (h) | 1 mg/kg | 0.1 mg/kg | 0.03 mg/kg | 0.01 mg/kg | mg/kg |
| 0 | 0.31 | 0.03 | 0.01 | 0.00 | 0.0003 |
| 0.17 | 1.65 | 0.17 | 0.05 | 0.02 | 0.0017 |
| 0.33 | 5.75 | 0.58 | 0.17 | 0.06 | 0.0058 |
| 0.5 | 8.08 | 0.81 | 0.24 | 0.08 | 0.0081 |
| 0.75 | 11.86 | 1.19 | 0.36 | 0.12 | 0.0119 |
| 1 | 15.57 | 1.56 | 0.47 | 0.16 | 0.0156 |
| 2 | 26.61 | 2.66 | 0.80 | 0.27 | 0.0266 |
| 4 | 32.64 | 3.26 | 0.98 | 0.33 | 0.0326 |
| 6 | 33.18 | 3.32 | 1.00 | 0.33 | 0.0332 |
| 8 | 34.91 | 3.49 | 1.05 | 0.35 | 0.0349 |
| 10 | 29.69 | 2.97 | 0.89 | 0.30 | 0.0297 |
| 12 | 28.40 | 2.84 | 0.85 | 0.28 | 0.0284 |
| 24 | 17.19 | 1.72 | 0.52 | 0.17 | 0.0172 |
| AUC | 294 | 29.38 | 8.81 | 2.94 | 0.29 |
| (h · nM) | | | | | |
| Cmax (nM) | 35 | 3.491 | 1.047 | 0.349 | 0.035 |

### Example 6

### Human Dose Prediction

Human dose prediction for clinical studies requires an estimate of human PK parameters important for defining the elimination T_{1/2} and the shape and magnitude of the plasma concentration vs time profile at a particular dose. These estimated parameters include Clearance, Volume of Distribution at Steady State (Vss), absorption rate constant (Ka), bioavailability (F) and dosing frequency. Human dose predictions also require some pharmacological evidence as to how exposure relates to efficacy (McGinnity, Collington, Austin & Riley, Current Drug Metabolism 2007 8 463-479).

For E1, human clearance was estimated from intrinsic clearance (Clint) data determined in human hepatocytes corrected to in vivo clearance by incorporation of the well-stirred model (Riley, McGinnity & Austin, Drug Metabolism & Disposition 2005 33(9) 1304-1311). Human Vss was taken to be the average of that observed in rat, guinea pig and dog is in vivo (35.5, 10.2 and 27 L/kg, respectively) plus that derived by simple allometry across rat and dog (5.7 L/kg), while Fabs (fraction absorbed) was assumed to be the average of that observed in rat and dog (83% and 70%, respectively). This Fabs parameter was then adjusted to bioavailability (F) by correction for hepatic extraction. The absorption rate (Ka) was set to ensure that Tmax was 1 h for E1, a relatively conservative value given that observed pre-clinically, and should equally result in a conservative Cmax value for use in calculation of margins with respect to safety studies. The clinical dosing frequency desired was assumed to be once daily.

Having defined the shape and magnitude of the human plasma concentration vs time curve, the final step was to adjust the dose so that plasma concentrations are attained that are likely to achieve efficacy. In mouse PD studies, efficacy against MMP down-regulation and joint swelling was observed at very low doses (as low as 0.0001 mg/kg). It was impossible to quantify the compound at these dose levels; consequently plasma concentrations observed at a higher oral dose (about 1 mg/kg) were extrapolated down to that likely at efficacious doses, assuming linear kinetics between the two. Finally, in the absence of a real PK/PD relationship in the mouse, it was assumed that efficacy in humans would be related to plasma AUC. Consequently, the human dose was adjusted to give, in conjunction with the estimated human PK parameters defined above, a human AUC value similar to that observed in mouse at efficacious doses.

### Results

Using the method described above, a human predicted dose of about 0.0001 milligrams of E1 per kilogram of bodyweight per day was obtained.

### CONCLUSION

By virtue of its ability to decrease levels of MMP13 and its aggrecanase inhibitory activity, N-methyl-1-{5-[(2R)-3H-spiro[4-azabicyclo[2.2.2]octane-2,2′-furo[2,3-b]pyridin]-5′-yl]-2-thienyl}methanamine (E1) is likely to be an effective inhibitor of cartilage degradation and thereby of use in the treatment or prophylaxis of OA. Furthermore, the reduction in levels of MMP13 observed when E1 is administered at doses as low as 0.0001 mg/kg and the human dose prediction of 0.0001 milligrams per kilogram of bodyweight per day indicate that E1 will likely be effective in the treatment or prophylaxis of OA at concentrations much lower than would otherwise be expected. Consequently, the risk of developing serious side effects when treating patients with E1 should be reduced.

### REFERENCES

Bau B, Gebhard P M, Haag J, Knorr T, Bartnik E, Aigner T. Relative messenger RNA expression profiling of collagenases and aggrecanases in human articular chondrocytes in vivo and in vitro. Arthritis Rheum. October; 46(10):2648-57 (2002).
Bove S E, Laemont K D, Brooker R M, Osborn M N, Sanchez B M, Guzman R E, Hook K E, Juneau P L, Connor J R, Kilgore K S. Surgically induced osteoarthritis in the rat results in the development of both osteoarthritis-like joint pain and secondary hyperalgesia. Osteoarthritis Cartilage. October; 14(10): 1041-8 (2006).
Karahan S, Kincaid S A, Kammermann J R, Wright J C. Evaluation of the rat stifle joint after transection of the cranial cruciate ligament and partial medial meniscectomy. Comp Med.; 51(6):504-12 (2001).
Kawashima K, Yoshikawa K, Fujii Y X, Moriwaki Y, Misawa H. Expression and function of genes encoding cholinergic components in murine immune cells. Life Sci. [Epub ahead of print] (2007).
Knauper, V., Lopez-Otin, C., Smith, B., Knight, G., & Murphy, G. Biochemical characterization of human collagenase-3. J Biol Chem. 271, 1544-1550. (1996).
Kurzen H, Wessler I, Kirkpatrick C J, Kawashima K, Grando S A. The non-neuronal cholinergic system of human skin. Horm Metab Res. 39(2): 125-35 (2007).
Lark M. W., E. K. Bayne, J. Flanagan, C. F. Harper, L. A. Hoerner, N. I. Hutchinson, I. I. Singer, S. A. Donatelli, J. R. Weidner, H. R. Williams, R. A. Mumford & L. S. Lohmander: Aggrecan degradation in human cartilage. Evidence for both matrix metalloproteinase and aggrecanase activity in normal, osteoarthritic, and rheumatoid joints. J Clin Invest 100, 93-106 (1997).
Lips K S, Luhrmann A, Tschernig T, Stoeger T, Alessandrini F, Grau V, Haberberger R V, Koepsell H, Pabst R, Kummer W. Down-regulation of the non-neuronal acetylcholine synthesis and release machinery in acute allergic airway inflammation of rat and mouse. Life Sci.; [Epub ahead of print] (2007).
Mitchell, P. G., Magna, H. A., Reeves, L. M., Lopresti-Morrow, L. L., Yocum, S. A., Rosner, P. J., Geoghegan, K. F., & Hambor, J. E.; Cloning, expression, and type II collagenolytic activity of matrix metalloproteinase-13 from human osteoarthritic cartilage. J Clin Invest. 97, 761-768. (1996).

What is claimed is:

1. A method of treatment or prophylaxis of osteoarthritis, comprising administering to a patient in need thereof a therapeutically effective amount of a compound of Formula (I), or a pharmaceutically acceptable salt thereof:

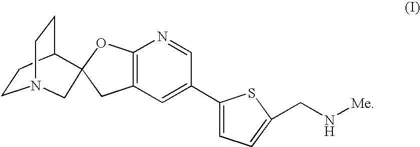

2. A method of treatment or prophylaxis of osteoarthritis, comprising administering to a patient in need thereof a therapeutically effective amount of a compound of Formula (IA), or a pharmaceutically acceptable salt thereof:

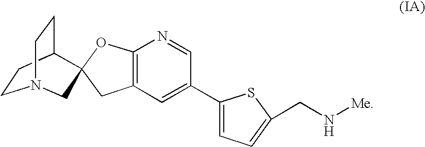

3. A method of treatment or prophylaxis of osteoarthritis according to claim 1, wherein the compound of Formula (I), a stereoisomer or a pharmaceutically acceptable salt thereof, is administered orally.

4. A method of treatment or prophylaxis of osteoarthritis according to claim 1, wherein the compound of Formula (I), a stereoisomer or a pharmaceutically acceptable salt thereof, is administered by intra-articular injection.

5. A method of treatment or prophylaxis of osteoarthritis according to claim 1, wherein the therapeutically effective amount comprises at least one dose of a compound of Formula (I), a stereoisomer or a pharmaceutically acceptable salt thereof, in the range of 0.00001 to 0.1 mg/kg of body weight per day.

6. A method according to claim 5 wherein the dose is in the range of 0.0001 to 0.01 mg/kg of body weight per day.

7. A method of treatment or prophylaxis of osteoarthritis according to claim 2, wherein the compound of Formula (IA), a stereoisomer or a pharmaceutically acceptable salt thereof, is administered orally.

8. A method of treatment or prophylaxis of osteoarthritis according to claim 2, wherein the compound of Formula (IA), or a pharmaceutically acceptable salt thereof, is administered by intra-articular injection.

9. A method of treatment or prophylaxis of osteoarthritis according to claim 2, wherein the therapeutically effective amount comprises at least one dose of a compound of Formula (IA), or a pharmaceutically acceptable salt thereof, in the range of 0.00001 to 0.1 mg/kg of body weight per day.

10. A method according to claim 9 wherein the dose is in the range of 0.0001 to 0.01 mg/kg of body weight per day.

11. A pharmaceutical composition comprising a therapeutically effective amount of a compound of Formula (I), or a pharmaceutically-acceptable salt thereof: 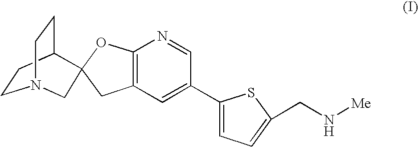
and a pharmaceutically acceptable carrier or diluent, for use in the treatment or prophylaxis of osteoarthritis.

12. A pharmaceutical composition comprising a therapeutically effective amount of a compound of Formula (IA), or a pharmaceutically-acceptable salt thereof: 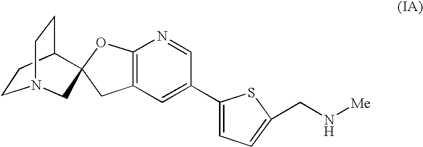
and a pharmaceutically acceptable carrier or diluent, for use in the treatment or prophylaxis of osteoarthritis.

13. A pharmaceutical composition comprising
0.0001 to 10 mg of a compound of Formula (I), or a pharmaceutically-acceptable salt thereof: 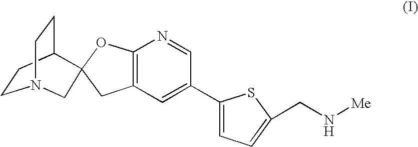
in combination with a pharmaceutically acceptable carrier or diluent.

14. A pharmaceutical composition comprising 0.0001 to 10 mg of a compound of Formula (IA), or a pharmaceutically-acceptable salt thereof: 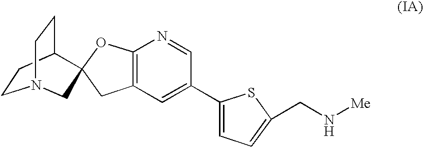
in combination with a pharmaceutically acceptable carrier or diluent.

15. A pharmaceutical composition according to claim 13 comprising 0.001 to 0.1 mg of a compound of Formula (I), or a pharmaceutically acceptable salt thereof.

16. A method of treatment or prophylaxis of osteoarthritis, which comprises administering to a patient in need thereof a pharmaceutical composition according to claim 13.

17. A pharmaceutical composition according to claim 14 comprising 0.001 to 0.1 mg of a compound of Formula (I), or a pharmaceutically acceptable salt thereof.

18. A method of treatment or prophylaxis of osteoarthritis, which comprises administering to a patient in need thereof a pharmaceutical composition according to claim 14.
